(19) United States
(12) Patent Application Publication
(10) Pub. No.: US 2026/0345678 A1
(43) Pub. Date: Nov. 5, 2026

(54) COMPOSITIONS, SYSTEMS, AND METHODS FOR COMBINATORIAL THERAPIES CONTAINING FUCOSYLATED CELLS AND CHEMOKINES AND/OR SCAFFOLDS FOR AUTOIMMUNE CONDITIONS AND REGENERATIVE MEDICINE

(71) Applicant: TARGAZYYME, INC., Carlsbad, CA (US)

(72) Inventors: David John Kyle, Davis, CA (US); Reid P. BISSONNETTE, Carlsbad, CA (US)

(21) Appl. No.: 19/487,183

(22) PCT Filed: May 24, 2024
(86) PCT No.: PCT/US2024/031026

§ 371 Date: Nov. 24, 2025

Related U.S. Application Data
(60) Provisional application No. 63/504,075, filed on May 24, 2023.

(51) Int. Cl. A61K 35/15; A61K 35/17; A61K 38/19; A61L 27/20; A61L 27/38; A61L 27/54; A61P 37/02; C12N 5/0775; C12N 9/10

(52) U.S. Cl. CPC A61K 35/15; A61K 35/17; A61K 38/195; A61L 27/20; A61L 27/3834; A61L 27/54; A61P 37/02; C12N 5/0663; C12N 9/1051; A61L 2300/426

(57) ABSTRACT

Compositions, medicaments, systems, and kits are disclosed that include at least one ex vivo fucosylated cell type, at least one chemokine, and/or at least one matrix scaffold. Methods of producing and using the compositions, medicaments, systems, and kits are also disclosed. In particular (but not by way of limitation), methods of treating at least one auto-immune disease and methods of repairing tissue damage due to injury or progressive disease are also disclosed.

### CROSS REFERENCE TO RELATED APPLICATIONS

The subject application claims benefit under 35 USC § 119(e) of U.S. Provisional Application No. 63/504,075, filed May 24, 2023. The entire contents of the above-referenced patent application(s) are hereby expressly incorporated herein by reference.

### BACKGROUND

Regenerative medicine is a new interdisciplinary field that aims to develop therapies to repair, regenerate, or replace damaged tissues and organs. It involves the use of various approaches, including stem cells, tissue engineering, and gene therapy, to restore normal function to damaged or diseased tissues and organs. The goal of regenerative medicine is to address the underlying cause of a disease or injury, rather than just treating the symptoms. Research in regenerative medicine is ongoing, and while some treatments have been developed and approved for clinical use, much work remains to be done before regenerative therapies become widely available for many conditions.

Autoimmune diseases are a group of disorders in which the immune system mistakenly attacks and damages healthy cells, tissues, and organs in the body. Normally, the immune system works to defend the body against foreign invaders, such as viruses and bacteria, and helps to identify and remove abnormal cells, such as cancer cells. In autoimmune diseases, however, the immune system cannot distinguish between healthy, normal cells and abnormal or foreign cells, resulting in chronic inflammation and tissue damage. The causes of autoimmune diseases are not fully understood, and the signaling differences between healthy cells and diseased cells have not been elucidated, but it is thought that genetic and environmental factors may play a role in their development. There is no cure for autoimmune diseases, but treatments are available to help manage symptoms and slow disease progression. However, there are many challenges and limitations associated with these treatments. For example, many medications used to treat autoimmune diseases can have side effects, such as nausea, vomiting, fatigue, and increased risk of infection. Some medications, such as corticosteroids, can also cause more serious side effects, such as bone loss and diabetes.

While many treatments can help to manage symptoms and slow disease progression, they are often not curative. In some cases, treatment may only provide temporary relief or may not be effective at all. Many of the medications used to treat autoimmune diseases can be expensive, particularly newer biologic therapies. This can limit access to treatment for some patients. Many autoimmune diseases require long-term treatment, which can be challenging for patients to manage and can increase the risk of side effects and complications. Many treatments for autoimmune diseases work by suppressing the immune system, which can increase the risk of infections and other complications. This can be particularly problematic for patients with compromised immune systems, such as the elderly or those with other underlying health conditions.

Immunosuppressive drugs are used to suppress the immune system, which can temporarily help to reduce inflammation and slow the progression of some autoimmune diseases. Monoclonal antibodies targeting specific proteins in the immune system that contribute to inflammation are biologic therapies for the treatment of some autoimmune diseases. Disease-Modifying Antirheumatic Drugs (DMARDs) can also help to slow the progression of certain autoimmune diseases, such as rheumatoid arthritis, while nonsteroidal anti-inflammatory drugs are commonly used to treat symptoms of autoimmune diseases such as joint pain and stiffness. Finally, making certain lifestyle changes such as maintaining a healthy diet, getting regular exercise, and managing stress can also help to manage symptoms of autoimmune diseases.

Stem cells are undifferentiated cells from which all other cells with specialized functions are generated. Under the right conditions in the body or a laboratory, stem cells divide to form more cells called daughter cells. The daughter cells become either new stem cells or differentiate into specialized cells with a more specific function, such as blood cells, brain cells, heart muscle cells, bone cells and cells in our immune system. No other cell in the body has the natural ability to generate new cell types. Hematopoietic stem cells (HSCs) have been used to treat various types of blood-borne cancers, including leukemia, lymphoma, and multiple myeloma, as well as other diseases such as blood disorders, genetic disorders, and metabolic disorders. In these cases, high doses of chemotherapy or radiation therapy are used to kill cancer cells, which also destroy the healthy stem cells. The Hematopoietic Stem Cell Transplant (HSCT) replaces the destroyed stem cells with new stem cells and helps the body recover. There are two main types of stem cell transplants: autologous and allogeneic. Autologous transplants involve using the patient's own stem cells, while allogeneic transplants involve using stem cells from a donor.

For a significant period of time during the HSCT process, the patient is severely immunocompromised, and many patients succumb to the lethal consequences of this process, even though they may have no more underlying disease. Key problems encountered in the immunocompromised phase of the HSCT process include elevated risks of bacterial, viral and fungal infection; internal uncontrolled bleeding; graft failure (GF); graft vs host disease (GvHD); and even growth of cancers due to lack of immune cells to combat the cancers.

Shortening the duration of the time when the patients are immuno-compromised can significantly improve patient outcomes, but it takes time to move the newly introduced stem cells out of the rapidly moving circulatory system into the tissues, and ultimately into the bone marrow where they will engraft and start the process of rebuilding the immune system. One solution that has been proposed is the ex vivo fucosylation of the stem cell surfaces (Popat, 2015, and US Patent Application Publication Nos. US 2011/0091434, US 2014/0161782, US 2017/0058261, and US 2019/0017023, all of which are incorporated herein by reference in their entirety). This fucosylation has been shown to improve the trafficking of the stem cells out of the circulatory system and into the bone marrow where they will differentiate into new immune cells. However, this process involves a one-time modification of the stem cells before they are infused into the patient, and as these stem cells divide, the fucosylated proteins on the cell surface are distributed to two daughter cells and are thereby diluted by 50% for each cell division.

### BRIEF DESCRIPTION OF THE DRAWINGS

FIG. 1 demonstrates that, in a GvHD animal model, fucosylated Treg-treated mice retained their body hair as opposed to their counterparts treated with Tregs that were untreated, who exhibited complete hair loss.

FIG. 2 demonstrates that fucosylated Tregs prevented rejection of a human skin graft in an NSG mouse human skin graft model.

FIG. 3 demonstrates that adoptive cell transfer (ACT) of fucosylated Tregs reduced development of Type 1 diabetes.

### DETAILED DESCRIPTION

Before explaining at least one embodiment of the present disclosure in detail by way of exemplary language, drawings, experimentation, results, and laboratory procedures, it is to be understood that the present disclosure is not limited in its application to the details of construction and the arrangement of the components set forth in the following description or illustrated in the drawings, experimentation, and/or results. The present disclosure is capable of other embodiments or of being practiced or carried out in various ways. As such, the language used herein is intended to be given the broadest possible scope and meaning, and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, and therapeutic applications.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, systems, kits, and methods disclosed herein can be made and executed without undue experimentation in light of the present disclosure. While the compositions, systems, kit, and methods have been described in terms of particular embodiments, it will be apparent to those of skill in the art that variations may be applied to the compositions, systems, kits, and methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example (but not by way of limitation), when associated with a particular event or circumstance, the term “substantially” means that the subsequently described event or circumstance occurs at least 80% of the time, or at least 85% of the time, or at least 90% of the time, or at least 95% of the time. In addition, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with,” “coupled to,” and “connected to” include both direct association/coupling/binding of two moieties to one another as well as indirect association/coupling/binding of two moieties to one another. When two moieties are indirectly associated/coupled/connected to one another, one or more intervening elements may be present therebetween (e.g., a spacer, linking moiety, etc.). Non-limiting examples of associations/couplings/bindings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “pharmaceutically acceptable” refers to compounds and compositions which are suitable for administration to humans and/or animals without undue adverse side effects, such as (but not limited to) toxicity, irritation, and/or allergic response, commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue. Non-limiting examples include a human, bovine, rat, mouse, dog, monkey, ape, goat, sheep, cow, or deer.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a subject/patient for therapeutic and/or prophylactic/preventative purposes.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

A “therapeutic composition,” “pharmaceutical composition,” or “medicament” refers to one or more agents that may be administered in vivo to bring about a therapeutic and/or prophylactic/preventative effect.

The term “regimen” refers to a protocol for dosing and timing the administration of one or more therapies (e.g., combinations described herein or another active agent such as an anti-cancer agent described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances, the term refers to active agents such as an anti-cancer agent described herein. The term “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or sufficient to exhibit a detectable therapeutic effect without undue adverse side effects (such as (but not limited to) toxicity, irritation, and allergic response) commensurate with a reasonable benefit/risk ratio when used in the manner of the present disclosure. The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given one active agent in conjunction with another active agent (i.e., one or more compositions or medicaments of the present disclosure). This concurrent therapy can be sequential therapy, where the patient is treated first with one composition and then the other composition, or the two compositions are given simultaneously.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The terms “administration” and “administering,” as used herein, will be understood to include all routes of administration known in the art. In addition, the compositions of the present disclosure (and/or the methods of administration of same) may be designed to provide delayed, controlled, or sustained release using formulation techniques which are well known in the art.

The term “coadministration” refers to administration of two or more agents (e.g., two or more agents of a combination described herein, or a combination described herein with another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The composition(s) of the present disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “pharmaceutically acceptable carrier or excipient” includes any carriers or excipients known in the art may be utilized in accordance with the present disclosure. For example (but not by way of limitation), a physiological compatible carrier (e.g., saline) that is compatible with maintaining the structure/activity of the active ingredient(s) when administered, and compatible with the desired mode of administration, may be utilized as the pharmaceutically acceptable carrier in accordance with the present disclosure. In addition, the active ingredient(s) may be mixed with excipients which are pharmaceutically acceptable and compatible with the active ingredient(s). Suitable excipients include, for example but not by way of limitation, water, saline, dextrose, glycerol, ethanol, and the like, or any combination thereof.

The term “adoptive cell therapy” or “ACT” refers to the transfer of cells into a patient. The cells may have originated from the patient or from another individual or from an inducible pluripotent stem cell (iPSC).

The terms “polypeptide” and “protein” are used interchangeably herein and refer to any molecule that includes at least two or more amino acids.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “enhance” refers to an increase or improvement in the function or activity of a protein or cell after administration or contacting with a combination described herein compared to the protein or cell prior to such administration or contact.

The terms “inhibition,” “inhibit,” “inhibiting” refer to a reduction in the activity, binding, or expression of a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating protein or enzyme activity or binding.

As used herein, “Current Good Manufacturing Practice” or “cGMP” refers to the Current Good Manufacturing Practice regulations enforced by the US Food and Drug Administration (FDA) or equivalent regulatory authorities in non-US countries. cGMP regulations provide for systems that assure proper design, monitoring, and control of manufacturing processes and facilities. Adherence to the cGMP regulations assures the identity, strength, quality, and purity of drug products by requiring that manufacturers of medications adequately control manufacturing operations. This includes establishing strong quality management systems, obtaining appropriate quality raw materials, establishing robust operating procedures, detecting and investigating product quality deviations, and maintaining reliable testing laboratories.

As used herein, the term “ex vivo expansion” or “expansion” refers to a method of growing a cell population in tissue culture that increases the number of cells in that population. Cells that have undergone ex vivo expansion are referred to as “expanded.”

As used herein, the term “fucosylation” refers to the treatment of a population of cells with an α1,3-fucosyltransferase and fucose donor under conditions that increase the ability of the cells to bind to a selectin or that increase the reactivity of the cells with an antibody known in the art to bind to sLeX including, but not limited to, the HECA-452 monoclonal antibody. Cells that have been treated with an α1,3-fucosyltransferase and fucose donor and then exhibit increased binding to selectins or to the HECA-452 monoclonal antibody or to another antibody specific for sLeX are referred to as being “fucosylated.” As used herein, “fucosylation” can also refer to the levels of sLeX present on a cell population.

Turning now to the inventive concepts, the present disclosure relates to the use of compositions, medicaments, systems, kits, and methods designed to provide a more effective treatment of autoimmune disease and rescue of tissue and organ function through regenerative cell therapy approaches. The concepts of the present disclosure are intended for use with child and adult patients or any individual who is in need of regenerating certain body structures, tissues, and organs to allow a patient to improve their quality of life. The present disclosure is relevant to individuals with autoimmune conditions, as well as those with structural damage to the body caused by disease or accident. The present disclosure related to methods of producing surface modifications to cells, such as but not limited to stem cells as well as circulating immune cells, and the use thereof to enhance the recovery of damaged tissues and to improve patient outcomes.

Regenerative medicine holds promise for the treatment of a wide range of conditions, including cardiovascular disease, neurodegenerative diseases, bone and joint disorders, diabetes, and cancer. It offers the potential to restore tissue and organ function, as well as to reduce the need for invasive surgeries and long-term drug treatments. Research in regenerative medicine is ongoing, but much work remains to be done before regenerative therapies become widely available for many conditions.

Autoimmune diseases are a group of disorders in which the immune system mistakenly attacks and damages healthy cells, tissues, and organs in the body. Normally, the immune system works to defend the body against foreign invaders, such as viruses and bacteria, and helps to identify and remove abnormal cells, such as cancer cells. In autoimmune diseases, however, the immune system cannot distinguish between healthy and foreign cells, resulting in chronic inflammation and tissue damage. There are more than 80 known autoimmune diseases, each of which can affect different parts of the body. Symptoms of autoimmune diseases can vary widely depending on the specific disease and the affected tissues and organs, but often include fatigue, joint pain and stiffness, skin rashes, and fever, and can end up in the death of the patient. The causes of autoimmune diseases are not fully understood, but it is thought that genetic and environmental factors may play a role in their development. There is no cure for autoimmune diseases, but treatments are available to help manage symptoms temporarily and slow disease progression. These treatments often involve medications to suppress the immune system, as well as lifestyle changes such as diet and exercise.

Multiple cell therapies are being employed to treat/reverse serious autoimmune diseases. They include hematopoietic stem cell transplantation (where regulatory T cells in the mononuclear cell population play a pivotal role), regulatory T cell transplantation, NK cell transplantation, and MSC therapies.

The present disclosure relates to the interface between regenerative medicine and the treatment of autoimmune disorders. Regenerative medicine and the treatment of autoimmune disorders are two rapidly evolving independent fields in medicine. The present disclosure provides new therapies for autoimmune disorders using cell therapies including (but not limited to) HSCT, Treg, NK, and MSC therapies and regenerative medicine. One non-limiting approach is to use stem cell modifications to efficiently regenerate damaged tissues and organs that have been attacked by the immune system in conditions such as, but not limited to the pancreas in Type 1 diabetes, the joints in rheumatoid arthritis, and the brain and neurological pathways in multiple sclerosis. A second approach described in detail herein uses regenerative medicine techniques and surface modified immune cells to modulate the immune system and prevent it from attacking healthy tissues. This can be done by using gene therapy, ex-vivo post-translational modification of immune cell surface proteins to modify the expression of certain genes involved in the immune response, and/or accelerating the homing process for the immune cells to the targeted tissues.

Fucosylated Tregs, Fucosylated MSCs, Fucosylated NK cells as individual cell therapy or in combination of mononuclear cells (MNCs) that are typically infused into the patients undergoing HSCT are also shown to improve their homing to sites of auto-immune disease attack to prevent/reduce incidence of auto-immune diseases such as Multiple Sclerosis, Lupus GVHD, T1D, Solid organ transplant rejection. By enabling multi-fold fucosylated Treg homing to the auto-immune disease sites as well as improving their residence time, immune balance is better restored with greater ratio of Tregs modulating the auto-reactivity of the T-cells that are attacking the patients' organs.

Overall, the interface between regenerative medicine using surface modified stem cells and the treatment of autoimmune disorders with surface modified immune cells is the genesis of novel effective therapies for these debilitating conditions.

### Cell Surface Modification in Regenerative Medicine

Although regenerative medicine holds great promise for the treatment of a wide range of diseases and injuries, there are several key limitations that still must be overcome. Regenerating complex tissues and organs is a difficult challenge, as it requires the coordination of many different types of cells and biological processes, and developing effective regenerative therapies for such tissues is still a major challenge. The safety of regenerative therapies is also an important consideration, as the use of stem cells and other biological materials can pose risks such as the development of tumors, immune reactions, and infections. Since the field is still new, there is a lack of standardization, therapies are expensive, and the use of embryonic stem cells raises ethical concerns for some people.

There are two important steps that allow stem cells and/or immune cells to home in on, and arrive at, a site of tissue damage in the body. If either step is delayed or dysfunctional, the immune cells may not arrive at the damaged site soon enough to neutralize whatever invasion is causing the damage, and the stem cells may also not arrive at the site of damage soon enough to repair it. Step 1 involves mobilizing the stem cells and/or the immune cells to effectively respond to the chemokines which are released from the injured tissues and act like target beacons for the immune cells. Stem cells are also able to sense and respond to signals from neighboring cells closer to the injured tissue. These signals can include (but are not limited to) growth factors and cytokines that activate specific cell signaling pathways that direct the stem cells towards the site of injury. Once closer, additional physical signals such as stiffness or elasticity of the tissues also come into play. Since the stem cells and immune cells are transported to the site of injury via the vascular system, the second step in the process involves the transit of these cells out of the fast-moving circulatory system and into the surrounding tissues. This can be achieved by a series of braking movements to specifically slow down and stop the stem cells in the fast-moving current so they can slip between endothelial cells and escape into the tissues (a process known as diapedesis). Both processes involve receptor/ligand interactions occurring on the surface of the stem cells, immune cells, and endothelial cells. These membrane protein receptors are generally glycosylated, which fine-tunes both the structure of the binding site for a specific ligand, and the subsequent intracellular signal that is generated by the binding of the ligand. Although these glycosylation patterns are genetically determined, the inventors have discovered that they can be modified ex vivo, and that certain specific changes can modify and enhance the characteristics of those cells.

In one non-limiting embodiment, the purified cells (such as, but not limited to, stem cells and/or immune cells) are activated ex vivo by contact with a fucosyltransferase (FUT) enzyme and its substrate GDP-fucose. In a particular (but non-limiting) embodiment, the purified cells are first expanded ex vivo (an adoptive cell therapy approach) from about 1- to about 50-fold, about 50- to about 100-fold, or from about 100- to about 500-fold before being fucose activated by fucosylation with the FUT and substrate. In either case the activation involves contact of the live cells with a fucosyltransferase (FUT) and GDP-fucose for a period of from about 5 to about 120 min, a period of from about 10 to about 90 min, or a period of from about 20 to about 30 min at a temperature of from about 10 °C to about 40 °C. In a particular (but non-limiting) embodiment, the time for fucosylation is about 30 min, and the temperature is about 37 °C.

In a particular (but non-limiting) embodiment, the FUT is selected from, but not limited to human FUT1, FUT2, FUT3, FUT4, FUT5, FUT6, FUT7, FUT8, FUT9, FUT10, and FUT11. In a more particular (but non-limiting) embodiment, the FUT is FUT6 or FUT7. In one non-limiting embodiment of the present disclosure, the FUT is a recombinant product of a FUT gene (such as, but not limited to, a human FUT gene) that is produced in a mammalian, insect, bacterial, yeast, or fungal expression system and purified prior to contact with the cells and GDP fucose. In a more particular (but non-limiting) embodiment, the nucleotide sequence of the membrane anchoring region of the FUT gene is removed in a way that makes the subsequently produced FUT enzyme soluble.

### Improvement of Treg Homing/Residence Time to Auto-Immune Disease Sites

Studies have shown that fucosylation enables multi-fold T-reg homing to auto-immune diseased sites as well as increasing the residence in diseased sites to reduce incidence as well as severity of auto-immune disease sites. The increased numbers of Tregs that are delivered to auto-immune disease sites as well as increased residence time restores the balance of Tregs and auto-reactive T-cells in the disease sites important for ameliorating the disease. The increased numbers of Tregs for sustained periods also enable improved ‘control’ of the auto-reactive T-cells to help prevent, reduce incidence and/or severity, and even stop the progression of the disease. The Tregs can be administered directly to the patient or as part of the mononuclear cell (MNC) population that is administered to patients as part of HSCT.

### Improvement of Stem cell and Immune Cell Homing to Injured Tissue

Studies have shown that fucosylation of hematopoietic stem cells (HSCs) from all HSCT sources (such as, but not limited to, peripheral blood, cord blood, and marrow) can improve their homing to bone marrow tissues. This is because the addition of fucose to the P-selectin glycoprotein ligand-1 (PSGL-1) on the surface of stem cells can facilitate the binding to P-selectin, (CD62P). P-selectin a cell adhesion molecule primarily expressed on the surface of activated endothelial cells lining blood vessels in the bone marrow microenvironment. This binding interaction is mediated by the interaction of P-selectin with specific carbohydrates on the fucosylated PSGL-1 protein, which allows the stem cells to adhere to the endothelial cells and subsequently extravasate into the bone marrow tissue. The binding of fucosylated PSGL-1 to P-selectin is a critical step in the homing of stem cells to bone marrow tissue. Once the stem cells have bound to P-selectin, they can then interact with other molecules and cells in the bone marrow microenvironment that facilitate their migration and differentiation into blood cells. In one non-limiting embodiment of the present disclosure, these other cells and molecules include, but are not limited to stromal cells, which provide support and structure to the bone marrow microenvironment, and produce cytokines and growth factors such as, but not limited to: Stem cell factor (SCF), Granulocyte colony-stimulating factor (G-CSF), Erythropoietin (EPO), Thrombopoietin (TPO), and Interleukins (e.g. IL-3, IL-6, and IL-11); chemokines (small signaling proteins that attract more stem cells to the bone marrow microenvironment and regulate their movement and localization within the tissue; and extracellular matrix proteins, which provide a scaffold for stem cells to adhere to and migrate through as they navigate the bone marrow microenvironment. Overall, the interaction between fucosylated PSGL-1 and P-selectin is an important mechanism by which stem cells, for example, are able to home to bone marrow tissue and contribute to the regeneration of blood cells.

Certain non-limiting embodiments of the present disclosure recognize that despite biochemical and functional differences, these observations from CBHSCs extend to other types of stem cells sourced from other human tissues including, but not limited to fetal, child, and adult blood, and other mesenteric tissues, and from differentiated somatic cells after they have been genetically reprogrammed as induced pluripotent stem cells (iPSCs) prior to ex vivo fucosylation. Specific stem cells in the present disclosure are prepared from peripheral blood (PBSCs), cord blood (CBSCs), mesenteric tissues (MSCs), or from somatic tissue that has been genetically reprogrammed as induced pluripotent stem cells (iPSCs). The stem cells of the present disclosure can be from any human or animal source. In a particular (but non-limiting) embodiment of the present disclosure, stem cells are harvested from the same person who is in need of treatment for the progressive disease or injury (i.e., an autologous transplant). In another particular (but non-limiting) embodiment of the present disclosure, stem cells are harvested from anyone other than the recipient (such as, but not limited to, a healthy individual who is a near relative of the patient or has certain characteristics that are compatible with the patient), and this is referred to as an allogeneic transplant.

Other non-limiting embodiments of the present disclosure also include but are not limited to the production and use of ex vivo fucosylated immune cells such as, but not limited to cytotoxic T-cells, regulatory T-cells, helper T-cells, NK-cells, B-cells, and dendritic cells.

Chemokines are important chemoattractants for stem cells and immune cells. They are small proteins that are classified into four main subfamilies based on the arrangement of conserved cysteine residues: CXC, CC, CX3C, and XC chemokines. Each subfamily has distinct structural features and functions. When a stem cell is contacted with a chemokine, several biochemical events can occur, leading to various cellular responses. The specific response depends on the type of stem cell, the chemokine receptor expressed, and the signaling pathways activated. Chemokine receptors are typically G protein-coupled receptors (GPCRs). Chemokine binding induces conformational changes in the receptor, leading to its activation. This activation triggers a series of intracellular signaling events including, but not limited to: (i) activating or inhibiting downstream signaling pathways that mediate different intracellular responses, (ii) generating intracellular second messengers such as (but not limited to) cAMP, IP3, and DAG which propagate the chemokine signal within the cell; (iii) activating signaling pathways including protein kinase cascades, phosphatidylinositol 3-kinase (PI3K)/Akt pathway, and mitogen-activated protein kinase (MAPK) pathways; (iv) modulation of gene expression by activating transcription factors or other regulatory proteins leading to changes in proliferation, differentiation, migration, or survival of stem cells; (v) enhancing stem cell migration to the source of a chemokine gradient; (vi) recruitment of stem cells to specific tissues or organs; (vii) modulation of stem cell differentiation pathways; and (vii) regulation of stem cell self-renewal.

Chemokine binding can be measured using various techniques that allow the detection and quantification of chemokine-receptor interactions such as (but not limited to) radioligand binding assays using radiolabeled chemokines. Alternatively, fluorescence-based binding assays with fluorescently labeled chemokines can be used.

In a non-limiting embodiment of the present disclosure, receptor activation on stem cells is measured using various experimental approaches that assess downstream signaling events or functional responses induced by receptor activation. These include but are not limited to: (i) Phosphorylation assays of intracellular signaling proteins, such as (but not limited to) kinases or transcription factors, by Western blots, or immunoprecipitation followed by immunoblotting; (ii) Calcium assays for the activation-induced increase in intracellular calcium concentration by detection and quantification of Ca using fluorescence microscopy or flow cytometry and calcium sensitive dyes such as (but not limited to) Fluo-4 or Fura-2; (iii) G protein activation assays such as (but not limited to) GTPγS binding assays or [35S]GTPγS autoradiography; (iv) Downstream gene expression analysis using Quantitative PCR (qPCR) or RNA sequencing (RNA-seq); and, more particularly (but not by way of limitation) (v) functional assays for cell migration, proliferation, differentiation, or survival. For example, chemotaxis assays can assess stem cell migration in response to a chemokine, proliferation assays can measure cell division, and differentiation assays can evaluate lineage-specific differentiation potential.

The chemokine CXCL12 (Stromal Cell Derived Factor 1; SDF-1) acts as a potent chemoattractant for immature and mature hematopoietic cells, and, thereby plays an important role in the homing of hematopoietic stem cells to a target tissue such as, but not limited to bone marrow, and mediates the survival and proliferation of colony-forming progenitor cells. CXCL12 binds to a highly glycosylated G-protein-coupled protein receptor (CXCR4) in the plasma membrane of hematopoietic stem and progenitor cells and immune cells. It is a major regulator of cellular trafficking. When CXCL12 binds to CXCR4, it triggers a cascade of intracellular signaling events that can improve the activation, migration, and survival of cells. CXCL12 is involved in the regulation of cell migration, development, and tissue homeostasis in various organs and tissues, including the bone marrow, lymph nodes, and brain. In bone marrow, CXCL12 plays a critical role in the homing and retention of hematopoietic stem and progenitor cells, which are essential for the maintenance of the blood system. In the lymph nodes, CXCL12 is involved in the regulation of immune cell trafficking and activation. In the brain, CXCL12 is involved in the development and maintenance of neural stem cells and in the regulation of neuronal migration and survival. In one non-limiting embodiment of the present disclosure, CXCL12 is added to the fucosylated stem cells to accelerate and amplify their activation prior to infusing them into the patient in need of a stem cell transplant.

The chemokine attractant CXCL10 (Interferon Gamma-Induced Protein 10; IP-10) binds to another G protein-coupled receptor (CXCR3) that is expressed on various immune cells, including T cells, natural killer cells, and dendritic cells. CXCR 3 is also expressed in hematopoietic stem cells, mesenchymal stem cells, and neural stem cells, but at a lower level of expression than in immune cells. Expression of CXCR 3 in mesenchymal stem cells can also be induced by inflammatory cytokines such as (but not limited to) interferon gamma and tumor necrosis factor alpha. When CXCL10 binds to CXCR3, it triggers a cascade of intracellular signaling events that can lead to the activation and migration of immune cells to sites of inflammation and injury, as well as modulating stem cell differentiation and function. Another non-limiting embodiment of the present disclosure relates to the improvement of stem cell homing by modifying the receptor binding site through ex vivo fucosylation to improve its ability to identify and home in on chemokine signals such as (but not limited to) CXCL12 and CXCL10 generated in injured cells or tissue whether this injury is due to a traumatic accident, a degenerative disease, an unexpected ischemic event, or encroachment by a rapidly dividing cancer cell mass. In a further non-limiting embodiment of the present disclosure, CXCL10 is added to the fucosylated stem cells to accelerate and amplify their activation prior to infusing them into the patient in need of a stem cell transplant.

One non-limiting embodiment of the present disclosure includes compositions and medicaments comprising ex vivo expanded and fucosylated stem cells in contact with one or more chemokine attractants such as, but not limited to CXCL12, CCL2, CCL5, CXCL10, and CXCL8 wherein such contact increase the expression of chemokine receptors including, but not limited to, CXCR4, CCR2, CCR5, CXCR3, and CXCR2 on the surface of immune cells such as, but not limited to, T-Cells, NK Cells, monocytes, and macrophages. Other non-limiting embodiments include compositions and medicaments comprising chemokines and stem cells such as but not limited to, hematopoietic stem cells, mesenchymal stem cells, neural stem cells, peripheral stem cells, pluripotent stem cells, and cord blood or placental stem cells. Additional non-limiting embodiments of the present disclosure involve the use of chemokines that have been fucosylated, through methods described herein and used in combination with stem cells or immune cells, and compositions and medicaments comprising one or more of fucosylated stem cells, fucosylated immune cells, and fucosylated chemokines.

### Treatment of Subjects with Autoimmune Conditions

In one non-limiting embodiment of the present disclosure, the compositions comprise a medicament for the treatment of at least one autoimmune condition including, but not limited to, one or more of any of the conditions selected from the group consisting of Acfatigueromegaly, Acquired aplastic anemia, Acquired hemophilia, Agammaglobulinemia, primary, Alopecia areata, Ankylosing spondylitis (AS), Anti-NMDA receptor encephalitis, Antiphospholipid syndrome (APS), catastrophic antiphospholipid syndrome (CAPS), Asherson's syndrome, Arteriosclerosis, Autoimmune Addison's disease (AAD), Autoimmune autonomic ganglionopathy (AAG), autoimmune dysautonomia, autoimmune gastrointestinal dysmotility (AGID), Autoimmune encephalitis (acute disseminated encephalomyelitis (ADEM), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, Autoimmune retinopathy (AIR), Autoimmune sudden sensorineural hearing loss (SNHL), Balo disease, Behçet's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Devic's disease/neuromyelitis optica (NMO), Type 1 Diabetes, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Glomerulonephritis, Goodpasture's syndrome/anti-GBM/anti-TBM Granulomatosis with polyangiitis (GPA)/Wegener's granulomatosis, Graves' disease/thyroid eye disease, Guillain-Barre syndrome (GBS), Hashimoto's thyroiditis/chronic lymphocytic thyroiditis/autoimmune thyroiditis, Henoch-Schonlein purpura/IgA vasculitis, Hidradenitis suppurativa, Hurst's disease 1 acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenia purpura/autoimmune thrombocytopenia, Inclusion body myositis, IgG4-related sclerosing disease (ISD), Interstitial cystitis, Juvenile idiopathic arthritis/Adult-onset Still's disease, Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Ménière's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pernicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Serpiginous choroidopathy, Sjogren's syndrome, Stiff person syndrome (SPS), Small fiber sensory neuropathy, Systemic lupus erythematosus (SLE), Subacute bacterial endocarditis (SBE), Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis (TM), Tubulointerstitial nephritis uveitis syndrome (TINU), Ulcerative colitis (UC), Undifferentiated connective tissue disease (UCTD), Uveitis |anterior/intermediate/posterior, Vasculitis, VEXAS Syndrome, Vitiligo, Vogt-Koyanagi-Harada syndrome (VKH), Parkinsons, ALS, dementia, Alzheimers, other neural diseases where disease progression is influenced by the onset of autoimmune diseases, and the like, as well as combinations thereof.

### Treatment of Subjects with Cancer

In one non-limiting embodiment, the compositions and medicaments are used in the treatment of hematopoietic cancers including, but not limited to the cancer treated is one or more of leukemia, lymphoma, and multiple myeloma including Acute myeloid (or myelogenous) leukemia (AML), Chronic myeloid (or myelogenous) leukemia (CML), Acute lymphocytic (or lymphoblastic) leukemia (ALL), Chronic lymphocytic leukemia (CLL), Hodgkin lymphoma, non-Hodgkin lymphoma (NHL), Light Chain Myeloma, Non-secretory Myeloma, Solitary Plasmacytoma, Extramedullary Plasmacytoma, Monoclonal Gammopathy of Undetermined Significance (MGUS), Smoldering Multiple Myeloma (SMM), Immunoglobulin D (IgD) Myeloma, Immunoglobulin E (IgE) Myeloma, and the like, as well as any combinations thereof.

In another non-limiting embodiment, the compositions and medicaments are used in the treatment of non-hematopoietic cancers (i.e., solid tumors) including, but not limited to one or more of prostate, skin, or ovarian cancer; cancers of non-lymphoid parenchymal organs including the heart, placenta, skeletal muscle and lung; breast cancer; cancers of the head and neck including various lymphomas, such as (but not limited to) mantle cell lymphoma, Non-Hodgkin B cell lymphoma, PTCL, adenoma, squamous cell carcinoma, laryngeal carcinoma, salivary carcinoma, thymomas and thymic carcinoma; leukemia; cancers of the retina; cancers of the esophagus; multiple myeloma; melanoma; colorectal cancer; lung cancer; cervical cancer; endometrium carcinoma; gallbladder cancer; liver cancer; thyroid follicular cancer; gastric cancer; non-small cell lung carcinoma; glioma; urothelial cancer; bladder cancer; prostate cancer; pancreatic cancer; renal cell cancer; infiltrating ductal carcinoma; and glioblastoma multiform.

### Treatment and Repair of Tissues of Subjects with Injuries or Progressive Diseases to Regenerate Normal Function

In one non-limiting embodiment, the compositions comprise a medicament for the repair of the human body from an injury or disease including, but not limited to bone fractures and defects, cartilage and ligament injuries, tendon and muscle injuries, skin wounds and burns, heart damage and cardiac disease, nerve damage and spinal cord injuries, liver disease, diabetes, kidney disease, eye disease and vision loss, muscle injuries, traumatic brain injuries, stroke, cerebral Palsy (ischemic brain injury), diabetic ulcers, corneal injuries, eye disorders such as (but not limited to) macular degeneration, hearing loss, dental injuries, lung injury and disease, peripheral arterial disease, neurological disorders such as (but not limited to) Parkinson's disease, multiple sclerosis, Alzheimer's disease, spinal muscular atrophy, muscular dystrophy, congenital heart defects, arthritis, autoimmune disorders, male and female infertility, erectile dysfunction, and, bone repair, nervous system repair, eye repair, skin repair, joint repair, liver repair, lung repair, pancreas repair, reproductive system repair, gastrointestinal repair, kidney repair, and circulatory system repair. In another non-limiting embodiment of the present disclosure, the composition and medicaments can be used on animals including, but not limited to, companion animals, performance animals, and animals maintained and managed for the production of food.

For the use of the instant compositions and medicaments, the stem cells can be sourced from human tissues including, but not limited to fetal tissues such as (but not limited to) cord blood and infant or child tissue, adult blood, and other mesenteric tissues, and from differentiated somatic cells after they have been genetically reprogrammed as induced pluripotent stem cells (IPSCs) prior to ex vivo fucosylation. Specific stem cells in the present disclosure are prepared from peripheral blood (PBSCs), cord blood (CBSCs), mesenteric tissues (MSCs), or from somatic tissue that has been genetically reprogrammed as induced pluripotent stem cells (iPSCs). The stem cells of the present disclosure can be from any human or animal source. In a particular (but non-limiting) embodiment of the present disclosure, the stem cells are harvested from the same person who is in need of treatment for the tissue injury. (i.e., an autologous transplant). If the stem cells are harvested from anyone other than the recipient (such as, but not limited to, a healthy individual who is a near relative of the patient or has certain characteristics that are compatible with the patient), it is referred to as an allogeneic transplant. The present disclosure can be practiced with both autologous and allogenic transplant procedures for humans and animals alike. According to the present disclosure, the harvested stem cells are purified prior to fucose activation by methods such as, but not limited to: 1) density gradient centrifugation; 2) Magnetic-activated cell sorting using magnetic beads coated with antibodies that bind to specific proteins on the surface of stem cells; 3) Fluorescence-activated cell sorting using fluorescent tags on antibodies that bind to specific proteins on the surface of the stem cells; and 4) Adhesion-based selection which allows stem cells to adhere to a surface or substrate that has been coated with specific proteins or extracellular matrix components that promote stem cell attachment.

One non-limiting embodiment of the present disclosure is the treatment of bodily injuries or damage to humans or other animals including, but not limited to companion animals (e.g., cats, dogs, horses, etc.), performance animals (e.g., sled dogs, racing horses, racing camels, etc.), and animals raised for food production (e.g., cows, sheep, goats, pigs, etc.). In one non-limiting embodiment of the present disclosure, the isolated and fucosylated stem cells used for the treatment of a bodily injury are injected directly into the recipient at or near the site of the injury. For example, in the case of a spinal cord injury, the stem cells can be injected into the spinal column in several different locations including, but not limited to, directly into the cerebral spinal fluid (CSF) surrounding the spinal cord through a lumbar puncture (Intrathecal Injection). The stem cells can then migrate to the site of injury in the spinal cord. In another non-limiting embodiment, the stem cells can be injected into the epidural space surrounding the spinal cord (Epidural Injection). Stem cells can promote tissue repair and functional recovery by releasing growth factors and other signaling molecules. In yet another non-limiting embodiment, the stem cells can be injected directly at the site of injury in the spinal cord. This may involve a surgical procedure to expose the spinal cord and deliver the stem cells. This method is more invasive than the intrathecal or epidural injection and requires precise localization of the injury site.

In another non-limiting embodiment, the isolated and fucosylated stem cells are first applied to a matrix for support, and the fucosylated stem cell rich matrix is contacted with the site of wound or injury by surgical means or through ultrasound guided injection. In one non-limiting embodiment of the present disclosure, the matrix is a hydrogel prepared from carbohydrate polymers. These carbohydrate polymers may include but are not limited to, hyaluronic acid, chitosan, alginate, dextran, fucoidan, and chondroitin. In a particular (but non-limiting) embodiment, the carbohydrate polymer is rich in fucose such as (but not limited to) fucoidan. Fucoidan is a complex polysaccharide that is extracted from various species of brown seaweed using various methods, such as (but not limited to) hot water extraction, enzymatic digestion, or acid hydrolysis. The extracted fucoidan is then purified to remove any impurities. It is a biocompatible material that is non-toxic and non-immunogenic. Its precise chemical structure can vary depending on the source and method of extraction. However, the main structural features of fucoidan are the presence of sulfated fucose residues, which are connected by glycosidic linkages. Its chemical structure is typically represented as a repeating disaccharide unit consisting of fucose (Fu) and sulfate (SO₄) groups. In one non-limiting embodiment, a scaffold-based delivery of stem cells would involve using a biomaterial scaffold to deliver stem cells to the site of injury. The scaffold provides a physical framework to which the stem cells attach and grow and can be designed to provide biochemical cues that encourage stem cell differentiation and tissue regeneration. This method involves surgically placing the scaffold at the site of injury and has the advantage of providing a more structured and controlled environment for stem cell growth and differentiation. In a particular (but non-limiting) embodiment, the one or more chemokines are contacted with the matrix-bound fucosylated stem cells prior to, contemporaneously with, or following the application of the scaffold to the injury site to further activate the cells through the binding of the chemokine to the chemokine receptor on the stem cell surface.

Fucoidan can be purified and processed into a suitable scaffold material in the form of a hydrogel, a water-swollen network of polymers that can provide a three-dimensional (3D) environment for stem cells to grow and differentiate. In a particular (but non-limiting) embodiment of the present disclosure, the fucoidan is mixed with phosphate buffered saline (PBS) to provide a mixture that is from about 70 wt % to about 99 wt % water content and cross-linked to provide a better scaffold for the stem cells. In a particular (but non-limiting) embodiment of the present disclosure, the matrix water content is from about 75 wt % to about 95 wt %; in a more particular (but non-limiting) embodiment, it is from about 80 wt % to about 90 wt %. The fucoidan can be crosslinked using a variety of methods, including, but not limited to, chemical crosslinking, physical crosslinking, or enzymatic crosslinking. Chemical crosslinking involves the use of a crosslinking agent, such as (but not limited to) glutaraldehyde or genipin, to form covalent bonds between fucoidan molecules. Physical crosslinking involves the use of physical forces, such as (but not limited to) temperature or pH changes, to induce the formation of physical crosslinks between fucoidan molecules. Enzymatic crosslinking involves the use of enzymes, such as (but not limited to) tyrosinase or peroxidase, to catalyze the formation of crosslinks between fucoidan molecules. In a particular (but non-limiting) embodiment, genipin, derived from the fruit of Gardenia jasminoides, is used as the cross-linking agent in the present disclosure, as it is biocompatible, provides stable crosslinking, and is not cytotoxic. Genipin also promotes cell adhesion, migration, and proliferation of stem cells.

The fucoidan hydrogel must be sterilized before use to prevent contamination. Sterilization can be achieved using various methods, such as, but not limited to, gamma irradiation or ethylene oxide sterilization. Once the fucoidan hydrogel has been prepared, it can be seeded with stem cells and used as a scaffold for stem cell transplantation. In one non-limiting embodiment of the present disclosure, stem cells are seeded onto the fucoidan hydrogel scaffold, either by direct injection into the scaffold or by allowing the cells to attach and proliferate on the scaffold surface. The seeded stem cells are then cultured on the fucoidan scaffold in a suitable culture medium such as, but not limited to, phosphate-buffered saline (PBS). The scaffold provides a supportive environment for the stem cells to differentiate into the desired cell type, such as (but not limited to) neurons or glial cells in the case of neural stem cells.

In a particular (but non-limiting) embodiment of the present disclosure, the stem cells are fucosylated according to the present disclosure and activated to differentiate into certain cell types prior to their incorporation in the hydrogel scaffold matrix. For example, where a stem cell-containing fucoidan hydrogel is to be applied to an injury such as (but not limited to) a torn Achilles tendon, the fucosylated stem cells can be pre-differentiated into the desired cell type, such as (but not limited to) tenocytes, which are the cells that make up the Achilles tendon prior to incorporation into the fucoidan hydrogel scaffold. Such a pre-differentiation process can be accomplished through processes such as, but not limited to, growth factor induction, mechanical stimulation, and co-culture with tenocytes. Growth factors, such as (but not limited to) transforming growth factor beta (TGF-β) and bone morphogenetic protein (BMP), can be added to MSC culture media to induce differentiation into tenocytes. These growth factors can promote the expression of tendon-specific genes and proteins, such as (but not limited to) collagen type I and III, tenascin-C, and scleraxis. MSCs can be subjected to mechanical stimulation, such as (but not limited to) cyclic stretching or compression, to induce tenogenic differentiation. Mechanical stimulation can activate signaling pathways involved in tendon development and promote the expression of tendon-specific genes and proteins. MSCs can also be co-cultured with tenocytes, which can provide cues and signals that promote differentiation into tenocytes. Co-culture with tenocytes can also facilitate cell-cell interactions and the exchange of paracrine signals, which can promote tenogenic differentiation.

In another non-limiting embodiment of the present disclosure, fucosylated immune cells including, but not limited to, platelets, cytotoxic T-cells, regulatory T-cells, helper T-cells, NK-cells, B-cells, and dendritic cells, can be included in the hydrogel matrix scaffold along with the fucosylated stem cells.

### EXAMPLES

To facilitate a more complete understanding of the present disclosure, examples are provided below. However, the scope of the present disclosure is not limited to specific embodiments disclosed in these examples, which are for purposes of illustration only.

The following examples are set forth below to illustrate the methods and results according to the disclosed subject matter. These samples are not intended to be inclusive of all aspects of the subject matter disclosed herein, but rather to illustrate representative methods and results. These examples are not intended to exclude equivalence and variations of the present disclosure that are apparent to one skilled in the art. The Examples are simply provided as one of various embodiments and are meant to be exemplary, not exhaustive.

### Example 1. Isolation and Expansion of Mesenchymal Stem Cells (MSCs) from Bone Marrow Cells

The donor is given local anesthesia to minimize discomfort, and the bone marrow is harvested from the iliac crest (hip bone) using a needle and syringe. The bone marrow is then processed to extract the stem cells from other components of the bone marrow using density gradient centrifugation, in which the bone marrow is layered on top of a density gradient solution and centrifuged, causing the different cell types to separate based on their density. The layer comprising MSCs is identified by the presence of specific cell surface markers, CD73 and CD105, and the layer comprising HSCs is identified by the presence of specific cell surface markers, CD34 and CD45. The isolated MSC fraction is then plated onto a culture dish containing Dulbecco's Modified Eagle Medium (DMEN) or alpha-MEM (minimum essential medium alpha), supplemented with fetal bovine serum (FBS) and antibiotics. The MSCs are then allowed to grow and expand in culture. The cells are incubated in a humidified atmosphere with 5% CO₂ at 37 °C, and the medium is changed every 2-3 days. After a 10-fold to 50-fold expansion, the MSCs are tested for their viability, sterility, and cell count by standard procedures to ensure that they are suitable for transplantation. The expanded MSCs are then cryopreserved in fetal bovine serum supplemented with DMSO and stored in a liquid nitrogen freezer until they are needed for transplantation.

### Example 2. Preparation and Testing of Recombinant Fucosyltransferase

A Fucosyltransferase FUT6 gene is isolated from a DNA library or genomic DNA using PCR amplification. The full genomic version of the gene contains a hydrophobic domain that anchors it in the membrane where it is normally located. The PCR product is purified and cloned into a baculovirus transformation vector wherein the amino acid residues 1-67 of the FUT6 gene, which comprise the signal peptide transmembrane region, are replaced by the sequence of the signal peptide of gp67 of the baculovirus for efficient secretion. The modified FUT6 gene is cloned into an appropriate vector for expression in CHO cells, such as (but not limited to) one that comprises the cytomegalovirus (CMV) promoter, and a selection marker to allow for selection of cells that have taken up the vector. The foreign gene-containing vector is introduced into the CHO cells using Lipofectamine as a transfection reagent. Different methods can be used for transfection, including electroporation, calcium phosphate transfection, and viral transduction. The transfected cells are then selected using the appropriate selection marker and screened for high-level expression of the foreign gene using techniques such as (but not limited to) Western blotting or ELISA. High-producing cell clones are selected and further characterized for protein expression level, stability, and other factors. The high-producing cell clones are grown in large-scale cell culture, and the recombinant Fucosyltransferase (rFUT) is secreted into the culture medium and purified using affinity chromatography or ion-exchange chromatography.

The final purified rFUT is then tested for enzymatic activity and its ability to fucosylate stem cells. Fucosylation of stem cells is measured using flow cytometry, which involves labelling cells with antibodies that recognize fucosylated proteins on the cell surface and passing the labelled cells through a flow cytometer, which detects and quantifies the fluorescent signal emitted by the labeled cells. The enzyme is considered usable in the present disclosure if it can produce a 4-fold or better signal in the stem cells vs. control when the enzyme is added along with its substrate GDP-Fucose in the cell culture medium at 37 °C within 30 minutes.

### Example 3. Fucosylation of MSC Cells

Fucosylation is the process of adding fucose sugars to glycoproteins. The materials required include the stem cells of interest from Example 1 and the rFUT enzyme of Example 2, as well as the FUT substrate, and Dulbecco's phosphate-buffered saline (DPBS) or other cell culture medium. Isolated, expanded, and fucosylated MSCs are transferred to DPBS or other cell culture medium until they reach around 70-80% confluency. The fucose donor substrate is prepared by dissolving GDP-Fucose in DPBS, to which is then added the expanded MSCs of Example 1 and the rFUT enzyme of Example 2. This mixture is allowed to incubate at 37 °C in a humidified incubator for 30-60 minutes with gentle mixing. After incubation, the fucose substrate mixture is removed and the fucosylated stem cells are washed three times with DPBS cell culture medium to remove any unbound enzyme or substrate and the fucosylated stem cells are ready to be added to a scaffold.

### Example 4. Preparation of Scaffolds with Fucosylated MSCs and Treatment of an Injured Achilles Tendo

A fucoidan scaffold is prepared starting with at least 85% purity fucoidan (e.g., Sigma-Aldrich; St Louis USA). The fucoidan hydrogel is prepared by adding Fucoidan to PBS to provide a hydrogel with about 85% water content. MSCs are then prepared and fucosylated and expanded as in Example 2 and activated to produce tenocytes by incubation with growth factor beta (TGF-β) and bone morphogenetic protein (BMP) for 1 hr at 37 °C. The fucosylated and activated MSCs are then added to the hydrogel mixture just before adding the genipin as a cross linking agent. Once the hydrogel is formed, it can be implanted into the injured area through minimally invasive techniques, such as (but not limited to) ultrasound-guided injection, or through open surgery. Following implantation, the patient is monitored and provided with appropriate care to ensure proper healing and integration of the hydrogel and stem cells. This may include immobilization of the affected area, physical therapy, and medication to manage pain and inflammation. The MSC-containing fucoidan hydrogel provides a scaffold that supports the attachment and proliferation of stem cells, while also promoting their differentiation into the desired cell type. Hydrogel also provides a hydrated and supportive microenvironment that can promote tissue repair and regeneration, and it slowly dissolves into the body tissues over 2-3 weeks following transplant.

### Example 5. Preparation of Scaffolds with Fucosylated MSCs and Treatment of Spinal Cord Injuries with Fucosylated Stem Cells

A patient with an acute or chronic Spinal Cord Injury (SCI) is evaluated by a medical expert to determine if they are eligible for stem cell therapy. This involves a thorough medical history, physical examination, and diagnostic tests such as (but not limited to) MRI scans. Stem cells are obtained from the patient's bone marrow and expanded in culture according to Example 1. The expanded SCs are then fucosylated according to Example 3 and at this stage are ready to be transplanted into the patient. The fucosylated stem cells (either alone or in combination with one or more chemokines, and/or alone or in combination with a matrix scaffold in which the fucosylated cells are dispersed) are transplanted into the patient's spinal cord by Intrathecal Injection, where the stem cells are injected directly into the cerebrospinal fluid (CSF) surrounding the spinal cord through a lumbar puncture. The stem cells then migrate to the site of injury in the spinal cord. After stem cell transplantation, the patient undergoes a rigorous rehabilitation program to promote functional recovery. This may involve physical therapy, occupational therapy, and/or other interventions aimed at improving mobility and quality of life.

### Example 6. Treating Neurological Deterioration in Patients with Multiple Sclerosis using Fucosylated MNCs Associated with Hematopoietic Stem Cell Transplantation

Multiple sclerosis (MS) is a chronic autoimmune disease that affects the central nervous system. There is currently no known cure for MS, but the goal of stem cell therapy for MS is to reset the immune system and prevent further damage to the nervous system. A patient with relapsing-remitting MS (RRMS) who had failed at least one disease-modifying therapy and with a minimum of one other medical condition that would increase the risks associated with hematopoietic stem cell transplant (HSCT) is identified by a qualified medical practitioner as being in need of a HSCT. MNCs are derived from peripheral blood, marrow, and/or cord blood and optionally expanded according to Example 1 and fucosylated according to Example 3. Prior to reintroducing the fuco-HCTs, the patient undergoes a conditioning regimen consisting of low-or medium-dose chemotherapy with a chemotherapeutic agent such as (but not limited to) cyclophosphamide. This regimen is designed to eliminate the existing immune system and make room for the transplanted stem cells. After completing the conditioning regimen, the patient receives a single infusion of a cocktail of stem cells that have been ex vivo fucosylated and CXCL12 (or other chemokine(s)) to reconstitute the immune system. The improved homing of the fuco-HSCs, amplified by the presence of CXCL12 (or other chemokine(s)), accelerates the recovery of the platelets and neutrophils to normal levels, minimizing the time the patient is at risk for infections and internal bleeding. The process also reduces the rate of occurrence of graft vs. host disease (GvHD) and generally improves the success rate of the engraftment of the new immune cells.

Most importantly the treated MNCs associated with HSCT includes a varied cell population that includes cell types such as (but not limited to) Tregs, MSCs, and NK cells. Fucosylation of the MNCs thus enables the resulting fucosylation of Tregs, MSCs, and NK cells to home to auto-immune disease sites to control inflammation. With fucosylated Tregs, fucosylation enables multi-fold more Tregs to reach disease sites such as the CNS with improved residence time, thereby improving the immune balance with improved control of the auto-reactive T-cells to prevent/reduce the incidence, severity, and/or progression of the auto-immune disease.

### Example 7. Treating an Autoimmune Disorder (Type 1 Diabetes) with Fucosylated Tregs and/or Fucosylated Mesenchymal Stem Cells

MSCs are a type of stem cell that can differentiate into a variety of cell types, including insulin-producing beta cells. In this example, MSCs and/or Tregs are harvested from a healthy donor (such as, but not limited to, from bone marrow) and expanded according to Example 1. The expanded MSCs and/or Tregs are then fucosylated as in Example 3. Fuco-MSCs and/or Fuco-Tregs (either with or without one or more chemokines) are then infused into the patient's bloodstream. Once inside the body, the MSCs migrate to the pancreas and differentiate into new beta cells.

### Example 8. Treating Lupus with Fucosylated Tregs, Fucosylated MNCs, Fucosylated NK Cells, and/or Mesenchymal Stem Cell (Fuco-MSC) Autologous Transplantation

Systemic Lupus with Erythematosus (SLE), commonly known as Lupus, is a chronic autoimmune disease that affects multiple organs and tissues in the body. Tregs and/or MSCs can regulate the immune system and reduce inflammation. In this example, MSCs are harvested from the patient's own bone marrow and expanded according to Example 1. They are then fucosylated according to Example 3 and infused into the patient's bloodstream as a cocktail with fucosylated CXCL12 and Fucosylated CXCL10. Once inside the body, the MSCs migrate to the sites of inflammation and release anti-inflammatory molecules to reduce inflammation and repair damaged tissues.

### Example 9. Non-Limiting Uses of Fuco-Cells for Repairing Damaged Tissues

The medicaments comprising ex vivo fucosylated cells of the present disclosure (either alone or in combination with at least one chemokine and/or a matrix scaffold) can be utilized in many other conditions, diseases, and conditions to repair damaged tissues and organs. Non-limiting examples of additional uses include the following.

Cardiac repair: use of fucosylated stem cells to repair damaged heart tissue after a heart attack or other cardiac events.

Bone repair: use of fucosylated stem cells to promote bone healing and regeneration, especially in cases of bone fractures, osteoporosis, and other bone-related disorders.

Nervous system repair: use of fucosylated stem cells as therapies for neurodegenerative diseases like Parkinson's, Alzheimer's, ALS, and the like as well as for spinal cord injuries and other nervous system disorders.

Skin repair: use of fucosylated stem cells to regenerate skin tissue in burn victims, patients with chronic wounds, and other conditions where skin regeneration is required.

Joint repair: use of fucosylated stem cells as treatments for joint degeneration, such as (but not limited to) in osteoarthritis.

Liver repair: use of fucosylated stem cells to regenerate liver tissue and treat liver diseases such as (but not limited to) cirrhosis and hepatitis.

Lung repair: use of fucosylated stem cells as therapies for lung diseases like cystic fibrosis, chronic obstructive pulmonary disease (COPD), and lung cancer.

Pancreatic repair: use of fucosylated stem cells as treatments for diabetes, by regenerating pancreatic beta cells that produce insulin.

Reproductive medicine: use of fucosylated cells in infertility treatments and regenerating reproductive organs.

Cancer treatment: use of fucosylated stem cells in cancer treatment to replace damaged bone marrow and restore the immune system after chemotherapy or radiation therapy.

### Example 10. Treatment of GvHD Effects with Fucosylated Regulatory T-cells

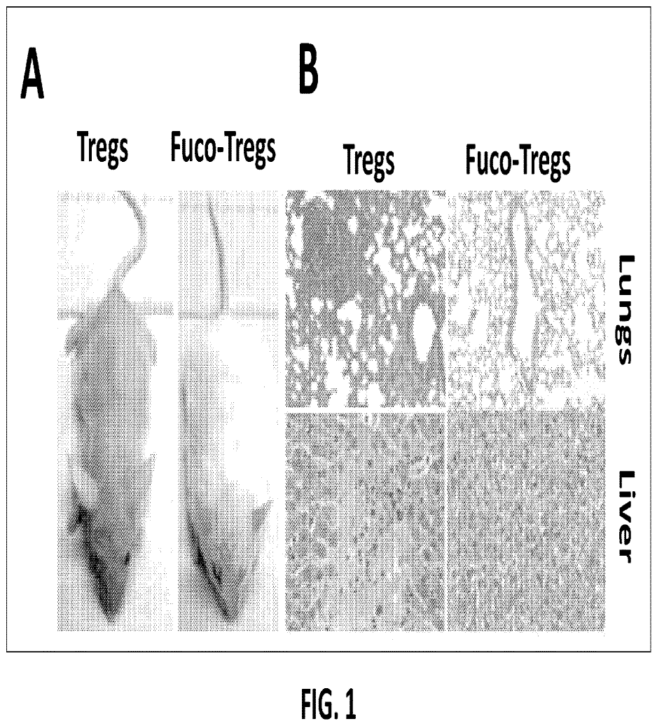

Graft versus Host Disease (GvHD) results in autoimmune attacks on skin and hair follicles, and the resultant hair loss is considered a classic symptom of GvHD. Therefore, the effects of fucosylated regulatory T-cells (Fuco-Tregs) on the symptoms of GvHD were investigated. As shown in Panel A of FIG. 1, mice that received Fuco-Tregs were able to retain their body hair, as opposed to complete hair loss observed in mice that received untreated Tregs. Panel B of FIG. 1 demonstrates that histopathological analyses of organ architecture showed extensive necrotic damage due to GvHD when untreated Tregs were administered, and this damage was absent in the Fuco-Treg-treated animals.

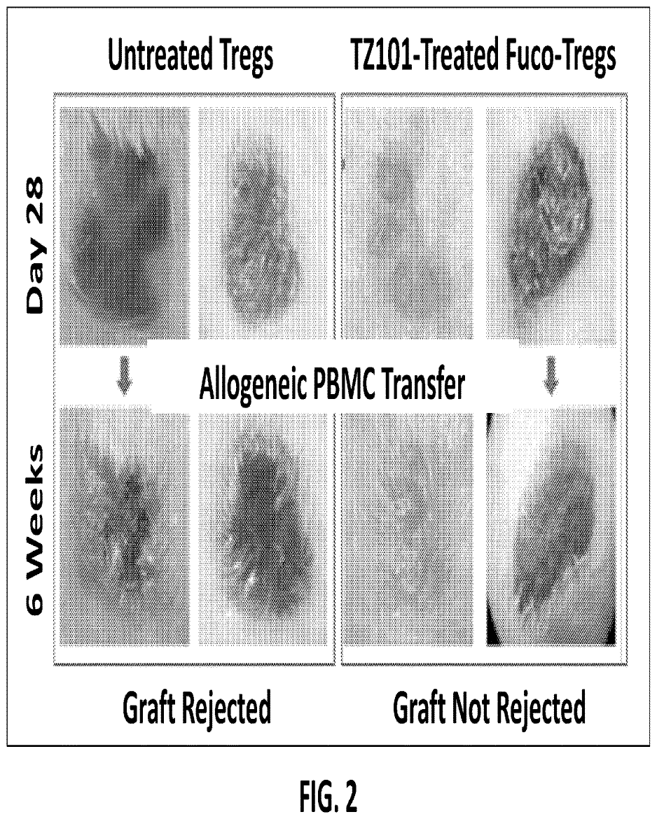

FIG. 2 demonstrates an analysis of autoimmune GvHD with an NSG (NOD-scid IL2rγ^{null}) mouse human skin graft model to study the possible abrogation of graft rejection with TZ101 (FUT6)-treated Fuco-Tregs. Human skin was grafted on NSG mice, where it healed well and was accepted by day 28. On day 29, the animals were injected with 10×10⁶ “recipient” human PBMCs allogeneic to the skin graft. The next day, the animals received either TZ101-fucosylated or unfucosylated 20×10⁶ polyclonally expanded autologous Tregs harvested from syngeneic mice spleens. At the end of 6 weeks, graft rejection was apparent in the unfucosylated Treg-infused mice, whereas administration of Fuco-Tregs prevented rejection of the skin graft.

### Example 11. ACT of Fuco-Tregs Reduced Development of Type 1 Diabetes (T1D)

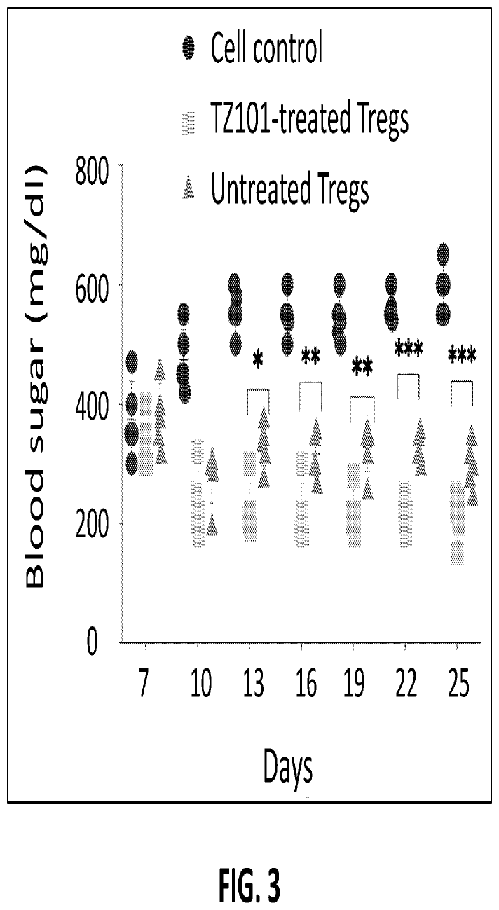

FIG. 3 demonstrates that Adoptive Cell Transfer (ACT) of Fuco-Tregs reduced T1D development. At day 0, naive RIP-OVA transgenic (Tg) mice (n=5) were i.v. adoptively transferred 3×10⁶ naive CD8⁺ T cells from OT-I T cell receptor Tg mice. At day 2, mice were i.p. challenged with VACV-OVA (2×10⁶ PFU/mouse). At day 7, mice were i.v. transferred with 3×10⁶ OVA-specific CD4⁺ CD25⁺ Tregs from OT-II TCR Tg mice treated with TZ101 or PBS control or CD4⁺CD25⁻CD4⁺ T cells (cell control). In the following days, blood sugar concentration was determined. Data shown are representative of 5 mice. **P<0.001; ***P<0.0001, 2-way ANOVA analysis. As can be seen, Fuco-Tregs were much more effective in reducing blood sugar levels and thereby reducing development of T1D than untreated Tregs alone.

### Non-Limiting Illustrative Embodiments

Illustrative embodiment 1. A medicament, comprising: a therapeutically effective amount of at least one ex vivo fucosylated cell type; and at least one chemokine.

Illustrative embodiment 2. A medicament, comprising: a therapeutically effective amount of at least one ex vivo fucosylated cell type; and at least one matrix scaffold in which the at least one ex vivo fucosylated cell type is disposed.

Illustrative embodiment 3. The medicament of Illustrative embodiment 1 or 2, wherein the at least one cell type that is ex vivo fucosylated is selected from the group consisting of stem cells, cytotoxic T-cells, regulatory T-cells, helper T-cells, NK-cells, B-cells, dendritic cells, macrophages, and combinations thereof.

Illustrative embodiment 4. The medicament of any of Illustrative embodiments 1-3, wherein the at least one ex vivo fucosylated cell type comprises ex vivo fucosylated stem cells.

Illustrative embodiment 5. The medicament of Illustrative embodiment 4, wherein the stem cells are selected from the group consisting of hematopoietic stem cells, mesenchymal stem cells, neural stem cells, peripheral stem cells, pluripotent stem cells, cord blood or placental stem cells, and combinations thereof.

Illustrative embodiment 6. The medicament of Illustrative embodiment 4 or 5, wherein the stem cells are sourced from embryonic tissues, fetal tissues, adult tissues and/or differentiated somatic cells after they have been genetically reprogrammed as induced pluripotent stem cells (iPSCs).

Illustrative embodiment 7. The medicament of any of Illustrative embodiments 1-6, wherein the cells are fucosylated ex vivo by contact with GDP-fucose and at least one of an effective fucosyltransferase (FUT), a recombinant fucosyltransferase (rFUT), or an active fragment of fucosyltransferase.

Illustrative embodiment 8. The medicament of any of Illustrative embodiments 17, wherein the at least one cell type that is ex vivo fucosylated comprises autologous cells.

Illustrative embodiment 9. The medicament of any of Illustrative embodiments 18, wherein the at least one cell type that is ex vivo fucosylated comprises allogeneic cells.

Illustrative embodiment 10. The medicament of any of Illustrative embodiments 1-9, wherein the at least one chemokine is from a chemokine family selected from the group consisting of the CXC, CC, CX3C, and XC families of chemokines.

Illustrative embodiment 11. The medicament of any of Illustrative embodiments 1-10, wherein the at least one chemokine is selected from the group consisting of CXCL12, CCL2, CCL5, CXCL10, CXCL8, and combinations thereof.

Illustrative embodiment 12. The medicament of any of Illustrative embodiments 1-11, wherein the at least one chemokine comprises CXCL12, CXCL2, CCL5, CXCL10, and CXCL8.

Illustrative embodiment 13. The medicament of any of Illustrative embodiments 1-12, wherein the at least one chemokine is present at a concentration in a range of from about 0.1 mg/ml to about 10 mg/ml.

Illustrative embodiment 14. The medicament of any of Illustrative embodiments 1-13, wherein at least one chemokine is ex vivo fucosylated.

Illustrative embodiment 15. The medicament of Illustrative embodiment 14, wherein the at least one chemokine is fucosylated ex vivo by contact with GDP-fucose and at least one of an effective fucosyltransferase (FUT), a recombinant fucosyltransferase (rFUT), or an active fragment of fucosyltransferase.

Illustrative embodiment 16. The medicament of any of Illustrative embodiments 1-15, wherein the at least one chemokine comprises at least one autologous chemokine.

Illustrative embodiment 17. The medicament of any of Illustrative embodiments 1-16, wherein the at least one chemokine comprises at least one allogeneic chemokine.

Illustrative embodiment 18. The medicament of any of Illustrative embodiments 1-17, wherein the at least one ex vivo fucosylated cell type comprises at least one ex vivo fucosylated stem cells, and wherein the medicament further comprises at least one immune cell type selected from the group consisting of cytotoxic T-cells, regulatory T-cells, helper T-cells, NK-cells, B-cells, dendritic cells, macrophages, and combinations thereof.

Illustrative embodiment 19. The medicament of Illustrative embodiment 18,wherein the at least one immune cell type is ex vivo fucosylated.

Illustrative embodiment 20. The medicament of Illustrative embodiment 18 or 19, wherein the at least one immune cell type comprises autologous immune cells.

Illustrative embodiment 21. The medicament of Illustrative embodiment 18 or 19, wherein the at least one immune cell type comprises allogeneic cells.

Illustrative embodiment 22. The medicament of any of Illustrative embodiments 18-21, wherein the at least one immune cell type is fucosylated ex vivo by contact with GDP-fucose and at least one of an effective fucosyltransferase (FUT), a recombinant fucosyltransferase (rFUT), or an active fragment of fucosyltransferase.

Illustrative embodiment 23. The medicament of any of Illustrative embodiments 1-22, further comprising at least one matrix scaffold.

Illustrative embodiment 24. The medicament of Illustrative embodiment 23, wherein at least a portion of the at least one matrix scaffold is cross-linked.

Illustrative embodiment 25. The medicament of Illustrative embodiment 23 or 24, wherein the at least one matrix scaffold comprises at least on ingredient selected from the group consisting of hyaluronic acid, chitosan, alginate, dextran, fucoidan, chondroitin, and combinations thereof.

Illustrative embodiment 26. The medicament of any of Illustrative embodiments 23-25, wherein the at least one matrix scaffold is a fucoidan-based scaffold.

Illustrative embodiment 27. The medicament of any of Illustrative embodiments 23-26, wherein the scaffold comprises a cross-linked fucoidan-based scaffold.

Illustrative embodiment 28. The medicament of any one of Illustrative embodiments 1-27, for use in the treatment of at least one autoimmune condition.

Illustrative embodiment 29. The medicament of Illustrative embodiment 28, wherein the at least one autoimmune condition is selected from the group consisting of Celiac disease, Crohn's disease (CD), Type 1 Diabetes, Eczema/Atopic Dermatitis, Endometriosis, Lupus, Lyme disease, Multiple sclerosis (MS), Chronic fatigue syndrome (CFS), Rheumatoid arthritis, Ulcerative colitis (UC), and combinations thereof.

Illustrative embodiment 30. The medicament of any one of Illustrative embodiments 1-29, for use in repair of tissue damage due to injury or progressive disease.

Illustrative embodiment 31. The medicament of Illustrative embodiment 30, wherein the tissue damage is a result of a condition selected from the group consisting of Bone fractures and defects, Cartilage and ligament injuries, Tendon and muscle injuries, Skin wounds and burns, Heart damage and cardiac disease, Nerve damage and spinal cord injuries, Liver disease, Diabetes, Lung disease, Kidney disease, Eye disease and vision loss, Bone fractures, Cartilage injuries, Tendon injuries, Ligament injuries, Muscle injuries, Spinal cord injuries, Traumatic brain injuries, Stroke, Cerebral Palsy (ischemic brain injury), Heart damage, Skin wounds and burns, Diabetic ulcers, Corneal injuries, Eye disorders such as (but not limited to) macular degeneration, Hearing loss, Dental injuries, Liver disease, Kidney disease, Lung injury and disease, Peripheral arterial disease, Neurological disorders such as (but not limited to) Parkinson's disease, Multiple sclerosis, Cerebral palsy, Alzheimer's disease, Spinal muscular atrophy, Muscular dystrophy, Congenital heart defects, Arthritis, Autoimmune disorders, Male and female infertility, Erectile dysfunction, Multiple sclerosis, and combinations thereof.

Illustrative embodiment 32. The medicament of Illustrative embodiment 30 or 31, wherein the tissue to be repaired is selected from the group consisting of cardiac, bone, nervous system, skin, joint, liver, lung, pancreas, reproductive system, kidney, cancerous tissue, and combinations thereof.

Illustrative embodiment 33. A kit, comprising: the medicament of any of Illustrative embodiments 1-32.

Illustrative embodiment 34. The kit of Illustrative embodiment 33, wherein the medicament is present in an IV bag for administration to a patient.

Illustrative embodiment 35. The kit of Illustrative embodiment 34, wherein the ex vivo fucosylated cells of the medicament is present in the IV bag at a concentration in a range of from about 106/100 ml to about 108/100 ml.

Illustrative embodiment 36. The kit of any of Illustrative embodiments 33-35, wherein the ex vivo fucosylated cells and the at least one chemokine are present in separate compartments of the kit.

Illustrative embodiment 37. The kit of any one of Illustrative embodiments 33-36, wherein the at least one chemokine comprises CXCL12, CXCL2, CCL5, CXCL10, and CXCL8.

Illustrative embodiment 38. The kit of any one of Illustrative embodiments 33-37, wherein the at least one chemokine is present at a concentration in a range of from about 0.1 mg/ml to about 10 mg/ml.

Illustrative embodiment 39. A method of treating a condition in a patient in need of treatment, the method comprising the steps of: administering to the patient the medicament of any one of Illustrative embodiments 1-32.

Illustrative embodiment 40. The method of Illustrative embodiment 39, wherein the medicament is infused to the patient at a dose of from about 10⁵ to about 10⁷ fucosylated cells/kg.

Illustrative embodiment 41. The method of Illustrative embodiment 39 or 40, wherein the method is further defined as a method of treating at least one autoimmune disease.

Illustrative embodiment 42. The method of Illustrative embodiment 41, wherein the at least one autoimmune condition is selected from the group consisting of Celiac disease, Crohn's disease (CD), Type 1 Diabetes, Eczema/Atopic Dermatitis, Endometriosis, Lupus, Lyme disease, Multiple sclerosis (MS), Chronic fatigue syndrome (CFS), Rheumatoid arthritis, Ulcerative colitis (UC), and combinations thereof.

Illustrative embodiment 43. The method of any of Illustrative embodiments 39-42, wherein the method is further defined as a method of repairing tissue damage due to injury or progressive disease.

Illustrative embodiment 44. The method of any of Illustrative embodiments 39-43, wherein the patient is a human.

Illustrative embodiment 45. The method of any of Illustrative embodiments 39-44, wherein the patient is a companion animal, performance animal, or an animal that is maintained and managed for the production of food.

Illustrative embodiment 46. A method of preparing a medicament, the method comprising the steps of: (1) harvesting cells from an autologous, allogeneic, or xenogeneic subject; (2) expanding the cells; (3) fucosylating at least a portion of the cells by contacting the cells with GDP-fucose and a fucosyltransferase or a functional fragment of a fucosyltransferase; and (4) combining the fucosylated cells with at least one chemokine to form the medicament.

Illustrative embodiment 47. A method of preparing a hydrogel-containing medicament for implantation into a patient, the method comprising the steps of: (1) harvesting cells from an autologous, allogeneic, or xenogeneic subject; (2) expanding the cells; (3) fucosylating at least a portion of the cells by contacting the cells with GDP-fucose and a fucosyltransferase or a functional fragment of a fucosyltransferase; (4) adding the fucosylated cells to a hydrogel mixture; and (5) adding a cross-linking agent to form the hydrogel-containing medicament.

Illustrative embodiment 48. The method of Illustrative embodiment 47, wherein the cells are stem cells, and wherein the method further comprises the step of activating the stem cells prior to addition to the hydrogel.

Illustrative embodiment 49. The method of claim 47 or 48, wherein the hydrogel mixture comprises water at a concentration in a range of from about 70 wt % to about 99 wt %.

Illustrative embodiment 50. The method of any of Illustrative embodiments 47-49, wherein the hydrogel mixture comprises fucoidan, and wherein the cross-linking agent comprises genipin.

Illustrative embodiment 51. A method of treating a patient in need thereof, the method comprising the steps of: (1) identifying a patient in need of a stem cell transplant (HSCT); (2) harvesting stem cells from the patient; (3) expanding the stem cells; (4) fucosylating at least a portion of the stem cells by contacting the stem cells with a fucosyltransferase and GDP-fucose; (5) forming a composition comprising the fucosylated stem cells and at least one chemokine; (6) exposing the patient to an immune ablation conditioning regimen comprising at least one immunosuppressant; and (7) infusing the composition comprising the fucosylated stem cells and at least one chemokine into the patient.

Illustrative embodiment 52. The method of any of Illustrative embodiments 46-51, wherein the fucosyltransferase or functional fragment thereof comprises a recombinant fucosyltransferase or functional fragment thereof, optionally produced in a mammalian, insect, bacterial, yeast, or fungal expression system, and optionally purified prior to use in step (3).

Illustrative embodiment 53. The method of any of Illustrative embodiments 46-52, wherein the functional fucosyltransferase fragment is soluble.

Illustrative embodiment 54. The method of any of Illustrative embodiments 46-53, wherein the functional fragment of fucosyltransferase is absent a membrane binding portion of fucosyltransferase.

Illustrative embodiment 55. The method of any of Illustrative embodiments 46-54, wherein the at least one cell type that is ex vivo fucosylated is selected from the group consisting of stem cells, cytotoxic T-cells, regulatory T-cells, helper T-cells, NK-cells, B-cells, dendritic cells, macrophages, and combinations thereof.

Illustrative embodiment 56. The method of any of Illustrative embodiments 46-55, wherein the at least one ex vivo fucosylated cell type comprises ex vivo fucosylated stem cells.

Illustrative embodiment 57. The method of Illustrative embodiment 56, wherein the stem cells are selected from the group consisting of hematopoietic stem cells, mesenchymal stem cells, neural stem cells, peripheral stem cells, pluripotent stem cells, cord blood or placental stem cells, and combinations thereof.

Illustrative embodiment 58. The method of any of Illustrative embodiments 56 or 57, wherein the stem cells are sourced from embryonic tissues, fetal tissues, adult tissues and/or differentiated somatic cells after they have been genetically reprogrammed as induced pluripotent stem cells (iPSCs).

Illustrative embodiment 59. The method of any of Illustrative embodiments 46-58, wherein the at least one cell type that is ex vivo fucosylated comprises autologous cells.

Illustrative embodiment 60. The method of any of Illustrative embodiments 46-59, wherein the at least one cell type that is ex vivo fucosylated comprises allogeneic cells.

Illustrative embodiment 61. The method of any of Illustrative embodiments 46-60, wherein the at least one chemokine is from a chemokine family selected from the group consisting of the CXC, CC, CX3C, and XC families of chemokines.

Illustrative embodiment 62. The method of any of Illustrative embodiments 46-61, wherein the at least one chemokine is selected from the group consisting of CXCL12, CCL2, CCL5, CXCL10, CXCL8, and combinations thereof.

Illustrative embodiment 63. The method of any of Illustrative embodiments 46-62, wherein the at least one chemokine comprises CXCL12, CXCL2, CCL5, CXCL10, and CXCL8.

Illustrative embodiment 64. The method of any of Illustrative embodiments 46-63, wherein the at least one chemokine is present at a concentration in a range of from about 0.1 mg/ml to about 10 mg/ml.

Illustrative embodiment 65. The method of any of Illustrative embodiments 46-64, wherein at least one chemokine is ex vivo fucosylated.

Illustrative embodiment 66. The method of Illustrative embodiment 65, wherein the at least one chemokine is fucosylated ex vivo by contact with GDP-fucose and at least one of an effective fucosyltransferase (FUT), a recombinant fucosyltransferase (rFUT), or an active fragment of fucosyltransferase.

Illustrative embodiment 67. The method of any of Illustrative embodiments 46-66, wherein the at least one chemokine comprises at least one autologous chemokine.

Illustrative embodiment 68. The method of any of Illustrative embodiments 46-67, wherein the at least one chemokine comprises at least one allogeneic chemokine.

Illustrative embodiment 69. The method of any of Illustrative embodiments 46-68, wherein the at least one ex vivo fucosylated cell type comprises at least one ex vivo fucosylated stem cells, and wherein the medicament further comprises at least one immune cell type selected from the group consisting of cytotoxic T-cells, regulatory T-cells, helper T-cells, NK-cells, B-cells, dendritic cells, macrophages, and combinations thereof.

Illustrative embodiment 70. The method of Illustrative embodiment 69, wherein the at least one immune cell type is ex vivo fucosylated.

Illustrative embodiment 71. The method of any of Illustrative embodiments 46-70, wherein the at least one immune cell type comprises autologous immune cells.

Illustrative embodiment 72. The method of any of Illustrative embodiments 46-71, wherein the at least one immune cell type comprises allogeneic cells.

Illustrative embodiment 73. The method of any of Illustrative embodiments 46-72, wherein the at least one immune cell type is fucosylated ex vivo by contact with GDP-fucose and at least one of an effective fucosyltransferase (FUT), a recombinant fucosyltransferase (rFUT), or an active fragment of fucosyltransferase.

Illustrative embodiment 74. The method of any of Illustrative embodiments 46-73, further comprising at least one matrix scaffold.

Illustrative embodiment 75. The method of Illustrative embodiment 74, wherein at least a portion of the at least one matrix scaffold is cross-linked.

Illustrative embodiment 76. The method of Illustrative embodiment 74 or 75, wherein the at least one matrix scaffold comprises at least on ingredient selected from the group consisting of hyaluronic acid, chitosan, alginate, dextran, fucoidan, chondroitin, and combinations thereof.

Illustrative embodiment 77. The method of any of Illustrative embodiments 74-76, wherein the at least one matrix scaffold is a fucoidan-based scaffold.

Illustrative embodiment 78. The method of any of Illustrative embodiments 74-77, wherein the scaffold comprises a cross-linked fucoidan-based scaffold.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

1. A medicament, comprising:
a therapeutically effective amount of at least one ex vivo fucosylated cell type; and
at least one chemokine.

2. The medicament of claim 1, wherein the at least one cell type that is ex vivo fucosylated is selected from the group consisting of stem cells, cytotoxic T-cells, regulatory T-cells, helper T-cells, NK-cells, B-cells, dendritic cells, macrophages, and combinations thereof.

3. The medicament of claim 1, wherein the at least one ex vivo fucosylated cell type comprises ex vivo fucosylated stem cells.

4. The medicament of claim 1, wherein the at least one cell type that is ex vivo fucosylated comprises autologous cells.

5. The medicament of claim 1, wherein the at least one cell type that is ex vivo fucosylated comprises allogeneic cells.

6. The medicament of claim 1, wherein the at least one chemokine is from a chemokine family selected from the group consisting of the CXC, CC, CX3C, and XC families of chemokines.

7. The medicament of claim 1, wherein the at least one chemokine is selected from the group consisting of CXCL12, CCL2, CCL5, CXCL10, CXCL8, and combinations thereof.

8. The medicament of claim 1, wherein at least one chemokine is ex vivo fucosylated.

9. The medicament of claim 1, wherein the at least one ex vivo fucosylated cell type comprises at least one ex vivo fucosylated stem cells, and wherein the medicament further comprises at least one immune cell type selected from the group consisting of cytotoxic T-cells, regulatory T-cells, helper T-cells, NK-cells, B-cells, dendritic cells, macrophages, and combinations thereof.

10. The medicament of claim 9, wherein the at least one immune cell type is ex vivo fucosylated.

11. The medicament of claim 1, further comprising at least one matrix scaffold.

12. The medicament of claim 11, wherein the at least one matrix scaffold is a fucoidan-based scaffold.

13. The medicament of claim 12, wherein the scaffold comprises a cross-linked fucoidan-based scaffold.

14-21. (canceled)

22. A method of treating a condition in a patient in need of treatment, the method comprising the steps of:
administering to the patient the medicament of claim 1.

23. The method of claim 22, wherein the medicament is infused to the patient at a dose of from about 10⁵ to about 10⁷ fucosylated cells/kg.

24. The method of claim 22, wherein the method is further defined as a method of treating at least one autoimmune disease.

25. The method of claim 24, wherein the at least one autoimmune condition is selected from the group consisting of Celiac disease, Crohn's disease (CD), Type 1 Diabetes, Eczema/Atopic Dermatitis, Endometriosis, Lupus, Lyme disease, Multiple sclerosis (MS), Chronic fatigue syndrome (CFS), Rheumatoid arthritis, Ulcerative colitis (UC), and combinations thereof.

26. The method of claim 22, wherein the method is further defined as a method of repairing tissue damage due to injury or progressive disease.

27. A method of preparing a medicament, the method comprising the steps of:
(1) harvesting cells from an autologous, allogeneic, or xenogeneic subject;
(2) expanding the cells;
(3) fucosylating at least a portion of the cells by contacting the cells with GDP-fucose and a fucosyltransferase or a functional fragment of a fucosyltransferase; and
(4) combining the fucosylated cells with at least one chemokine to form the medicament.

28-30. (canceled)

31. A method of treating a patient in need thereof, the method comprising the steps of:
(1) identifying a patient in need of a stem cell transplant (HSCT);
(2) harvesting stem cells from the patient;
(3) expanding the stem cells;
(4) fucosylating at least a portion of the stem cells by contacting the stem cells with a fucosyltransferase and GDP-fucose;
(5) forming a composition comprising the fucosylated stem cells and at least one chemokine;
(6) exposing the patient to an immune ablation conditioning regimen comprising at least one immunosuppressant; and
(7) infusing the composition comprising the fucosylated stem cells and at least one chemokine into the patient.
